(19) United States
(12) Patent Application Publication
(10) Pub. No.: US 2005/0090766 A1
(43) Pub. Date: Apr. 28, 2005

(54) Tube for blood collecting with a vacuum method

(76) Inventor: Renzo Montanari, Ravenna (IT)

Correspondence Address: MODIANO & ASSOCIATI, Via Meravigli, 16, Milano, 20123, IT

(21) Appl. No.: 10/691,589

(22) Filed: Oct. 24, 2003

(51) Int. Cl. A61B 5/00

(52) U.S. Cl. 600/573; 600/576

(57) ABSTRACT

A tube for collecting blood with a vacuum method that comprises a stopper and a blood collection needle arranged at an end of the tube for piercing insertion in the stopper. An annular lip is provided at an external region of the stopper that forms a groove surrounding the needle piercing point.

### BACKGROUND OF THE INVENTION

Blood to be analyzed is currently collected almost exclusively with sterile evacuated tubes which, by avoiding the use of classic syringes, make these collections quicker, less onerous and hygienically safer. This kind of tube is sealed by a rubber stopper, through which one end of a hollow needle is inserted at collection time. The needle is fixed through the bottom of a clear plastic cylinder that accommodates the tube to be filled, so as to have one end that is external to the cylinder and, when inserted in the vein of the patient, allows the transfer of the blood directly into the evacuated tube.

For hygiene reasons and to allow the handling of this implement with the necessary safety, the portion of needle that protrudes externally through the bottom of the plastic cylinder is protected by a cap to be manually removed when insertion in the vein is performed. The portion of said needle that must be inserted in the tube is instead enclosed in an elastic sheath, which protects the needle against external contaminations. Said sheath gathers up along said needle during its insertion in the rubber stopper of the tube and extends automatically with an opposite motion when the tube is extracted, so as to cover again the end of the needle at the end of the collection and prevent the dispersion of blood outside the tube.

However, at the end of the collection, the very act of extracting the needle from the rubber stopper causes, by entrainment, the outflow of a drop of blood, facilitated by the initial fluidity of the blood, the temperature of which is relatively high since it is substantially equal to the body temperature of the patient.

Obviously, the probabilities of contamination increase if the operator assigned to collections is particularly overworked and perhaps when, in having to collect a plurality of blood samples from each patient, he is forced to act rapidly and with the consequent risk of impacts that facilitate the outflow of said drops of blood from the stopper of the tubes.

### SUMMARY OF THE INVENTION

The aim of the present invention is to obviate the described severe drawback and to avoid the associated risks without however changing the usual collection operations, their cost and their execution times.

This aim is achieved with a tube provided with an improved stopper whose characteristics are defined in the appended claims.

### BRIEF DESCRIPTION OF THE DRAWINGS

The characteristics and advantages of the tube according to the invention are described hereinafter with the aid of two drawings, which illustrate only by way of non-limitative example:

FIG. 1, which is an exploded side view of an evacuated tube and of a known blood collection implement, shown in partial cross-section;

FIGS. 2 and 3, which are respectively an axial view and a longitudinal sectional view of the new improved stopper;

FIG. 4, which is a longitudinal sectional view of the assembly constituted by the collection implement and by the evacuated tube with the new improved stopper;

FIG. 5, which is a longitudinal sectional view, taken after collection and after removing the appropriately provided needle, of the tube with the new improved stopper, rested on a horizontal surface.

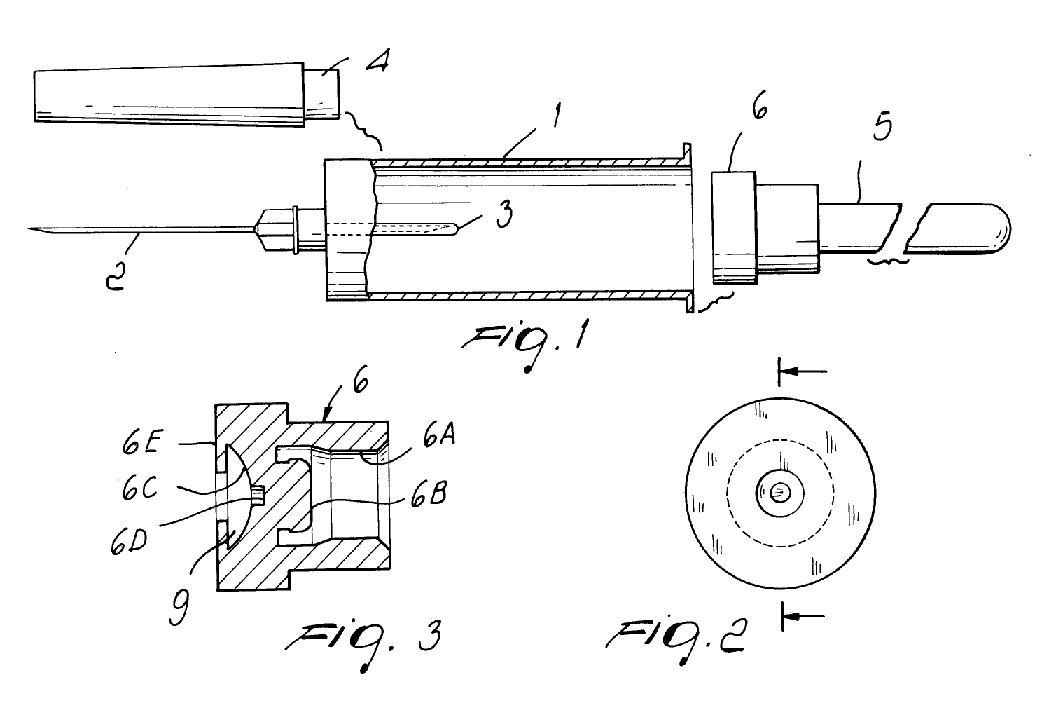

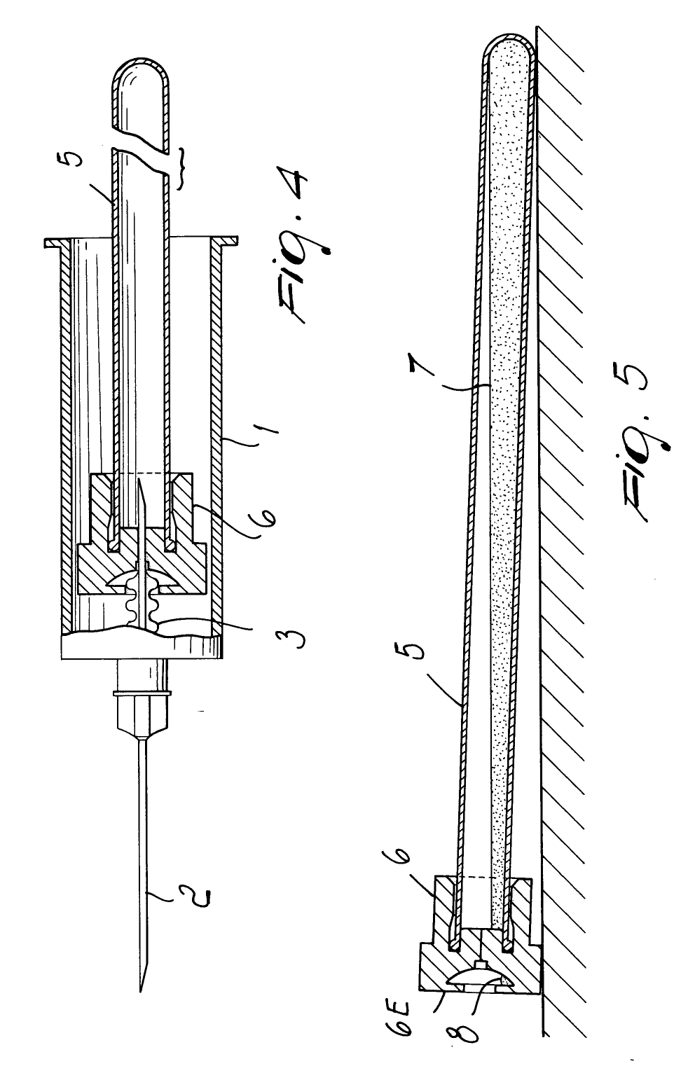

### DESCRIPTION OF THE PREFERRED EMBODIMENTS

In the figures, the reference numerals 5 and 6 designate respectively a tube and its stopper. The reference numeral 2 designates a hollow needle of an implement normally used with the evacuated tube to collect blood samples to be analyzed. The hollow needle 2 is axially fixed through the bottom of a clear plastic cylinder 1 and comprises two pointed opposite ends. One end protrudes outside, where it is protected by a rigid cap 4 that is applied to it and is to be removed manually upon insertion in the vein. The other end protrudes inside the cylinder 1, where it is instead completely covered by an elastic sheath 3. The sheath 3 is elastic, so that it can gather up when the needle is inserted in the stopper 6 of the tube 5 and can extend automatically during extraction from the stopper. In this manner, the sheath 3 protects the needle against external contaminations and prevents the blood from flowing out when the needle has not yet been inserted in the tube.

The improved stopper 6 is made of rubber or other suitable material and has a collar 6A and a concentric cylindrical protrusion 6B inside said collar. A seat remains between the collar 6A and the protrusion 6B, and the mouth of the tube engages therein so as to provide a double seal, i.e. on the outside and also on the inside of the mouth.

A cavity 6C shaped like a spherical dome is formed externally with respect to the stopper 6. A central hollow 6D is provided in the center of the cavity 6C and acts as a guide for the collection needle to be inserted. An annular lip 6E protrudes toward the center from the edge of the cavity 6C and forms, together with the cavity 6C, an annular groove 9 or undercut that is adapted to retain, even in case of impact of the tube on the supporting surface, any drop of blood 8 (FIG. 5) entrained externally, after taking the blood sample 7, by the extraction of the needle 2 from the stopper 6.

Further, the annular lip 6E prevents the operator from making accidental contact with the blood collected in the groove during subsequent handling of the tube.

Clearly, the example shown and described does not exclude the possibility of other embodiments suitable for the indicated purposes and comprised within the scope of the appended claims.

Thus, for example, the cavity 6C can be omitted and the groove 9 for collecting any blood drop entrained externally by the needle during its extraction can be formed between the annular lip 6E and the front face of the stopper.

1. A tube for collecting blood with a vacuum method, comprising a stopper and a blood collection needle arranged at an end of the tube so as to be insertable in a piercing point of said stopper for piercing therethrough, and wherein said stopper is provided with an annular lip located at an external region thereof, said lip forming a groove, said groove surrounding the piercing point of said needle in said stopper.

2. The tube of claim 1, wherein said stopper has an external cavity with an edge, said annular lip protruding inward from said edge so as to form in said cavity the annular groove that surrounds the piercing point of said needle in said stopper.
